(19) United States
(12) Patent Application Publication
(10) Pub. No.: US 2005/0165238 A1
(43) Pub. Date: Jul. 28, 2005

(54) Oxidation process for the preparation of intermediates useful in the synthesis of diarylpyridines

(76) Inventors: Vincenzo Cannata, Sasso Marconi (IT); Giorgio Soriato, Caldiero (IT); Massimo Verzini, Caldiero (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/499,321

(22) PCT Filed: Dec. 12, 2002
(86) PCT No.: PCT/EP02/14115

(30) Foreign Application Priority Data
Dec. 19, 2001 (IT) MI2001A002692

(51) Int. Cl. C07D 213/46

(52) U.S. Cl. 546/315

(57) ABSTRACT

An oxidation process for the preparation of intermediates useful in the synthesis of diarylpiridines is described. The oxidation of the thioether of formula III to give the sulfone of formula II is is carried out with a mixture of peracetic acid and hydrogen peroxide as oxidant, in the presence of methanesulphonic acid and in the absence of a catalylst.

The present invention concerns an oxidation process for the preparation of intermediates useful in the synthesis of diarylpyridines and, more particularly, it concerns to an oxidation process for the preparation of intermediates useful for the synthesis of compounds of formula 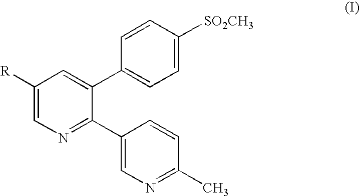
wherein R is chlorine, fluorine, bromine, iodine, CN or azohydrate; useful as cyclooxygenase-2 (COX-2) inhibitors.

The compounds of formula (1) are described in the patent application WO 98/03484 (Merck Frosst Canada Inc.).

An improved process for the synthesis of the compounds of formula (1), recently described in the patent application WO 99/15503 (Merck & Co., Inc.), is characterised by the synthesis of the compound of formula 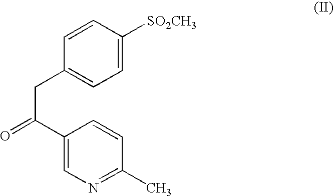
as key intermediate for the preparation of the COX-2 inhibitors of formula (I).

Substantially, the synthesis of the intermediates (II) consists of the reaction of a Grignard compound of formula 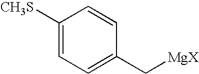
wherein X is chlorine, bromine or iodine;
and an amide (Weinreb amide) of formula 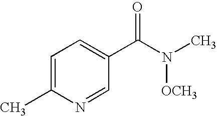
to give a compound of formula 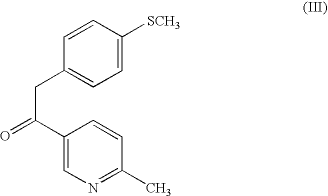
and its subsequent oxidation.

The oxidation reaction is carried out by using various oxidation systems like hydrogen peroxide, oxone® (2 KHSO₅.KHSO₄.K₂SO₄) or hydrogen peroxide/acetic acid, preferably by using oxone® or hydrogen peroxide in the presence of a catalyst, preferably Na₂WO₄, under acid conditions.

The presence of a second function which can be oxidized (the pyridine nitrogen atom) in the molecule to be oxidized causes the formation of compound II N-oxide as reaction by-product.

Using weak oxidation conditions does not represent a valid solution to the problem because, even if the formation of the N-oxide by-product is avoided, the oxidation of the sulphur atom is not complete with the consequence that sulphoxide derivatives are formed, which are difficult to be separated.

Recently two other patent applications have been published concerning the same topic: WO 01/29004 (Zambon Group S.p.A.) and WO 01/07410 (Lonza AG). In both the applications, the preferred conditions for carrying out the oxidation reaction of the last step, need the presence of a catalyst, in particular of a wolfram derivative (Na₂WO₄).

We have now surprisingly found that it is possible to carry out the above oxidation without the presence of a catalyst.

Accordingly, object of the present invention is a process for the preparation of the compound of formula 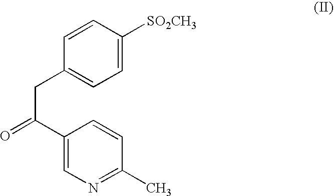
by oxidation of the compound of formula 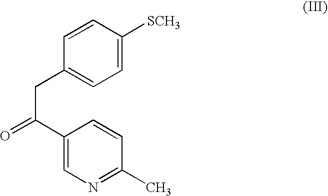
with an oxidant, in the presence of an acid, characterised by the fact that the oxidant is a mixture of peracetic acid and hydrogen peroxide and the acid is methanesulphonic acid.

The process object of the present invention is useful for the preparation of intermediates in the synthesis of COX-2 inhibitors.

The oxidant is used in excess and it consists of a mixture of peracetic acid and hydrogen peroxide in a suitable solvent.

A mixture of peracetic acid and hydrogen peroxide in acetic acid and water, commercially available (Oxystrong®-Ausimont) is particularly preferred.

The oxidant is used in slight excess with respect to the compound III, preferably in a molar ratio from 2.0 to 2.1.

Methanesulphonic acid is used in a slight excess with respect to compound III, preferably in a molar ratio from 1.1 to 1.4.

The oxidation process object of the present invention allows to selectively oxidise the methylthio group to the methylsulphonyl group with good yields without the use of a metal catalyst and with non-significant amounts of N-oxide as by-product.

Moreover, the process object of the present invention allows to isolate the final product without a further purification step and this is very advantageous by the industrial point of view.

Accordingly, the expert in the field can immediately recognise how the above reported advantages make the process of the present invention more convenient also by the economic and environmental viewpoints.

In order to better illustrate the present invention the following example on industrial scale is now given.

### Example 1

Into a reactor, equipped with mechanic stirrer, thermometer and condenser, 1-(6-methyl-3-pyridinyl)-2-(4-methylthio-phenyl)-ethanone (30 kg; 116.7 moles), acetic acid (45 kg) and methanesulphonic acid (13.6 kg; 141,2 moles) are charged.

The addition of methanesulphonic acid is hexothermic and the temperature is kept at 30-35° C. By keeping the solution at 35° C. Oxystrong® (28.1 kg, 240.4 moles; 65% titer as peracetic acid) is added. The reaction mixture is kept at 35° C. for 3-4 hours. The excess oxidant is removed by adding sodium thiosulfate pentahydrate and the cloudy solution is cleared by filtration on celite. The system is diluted with ethylacetate (93.3 kg; 103.7 l) and water (37.7 kg).

A 30% sodium hydroxide solution (36.5 kg; 28.1 l) is added in 1.5 hours up to a final pH of 4-4.5 letting the temperature raise spontaneously. Once reached the desired pH, the suspension is reflux heated (about 60-65° C.) for 30 minutes, cooled under stirring to about 20-25° C. and then centrifuged.

The solid is collected, washed with ethyl acetate, water and again with ethylacetate. After drying at 60° C. under vacuum up to constant weight, 1-6-methyl-3-pyridyl)-2(4-methylsulfphonylphenyl)-ethanone (30 kg; 88.6% molar yield) is obtained.

1) A process for the preparation of the compound of formula 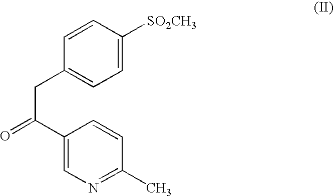
by oxidation of the compound of formula 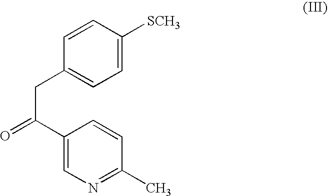
with an oxidant, in the presence of an acid, characterised in that the oxidant is a mixture of peracetic acid and hydrogen peroxide and the acid is methanesulphonic acid.

2) A process according to claim 1 wherein the oxidant is used in excess.

3) A process according to claim 1 wherein the oxidant is a mixture of peracetic acid and hydrogen peroxide in a suitable solvent.

4) A process according to claim 1 wherein as oxidant a mixture of peracetic acid and hydrogen peroxide in acetic acid and water is used.

5) A process according to claim 1 wherein methanesulphonic acid is used in a molar ratio from 1.1 to 1.4 with respect to compounds III.
